(19) United States
(12) Patent Application Publication
(10) Pub. No.: US 2024/0074810 A1
(43) Pub. Date: Mar. 7, 2024

(54) SYSTEMS AND METHODS FOR SURGERY PLANNING

(71) Applicant: SHANGHAI UNITED IMAGING INTELLIGENCE CO., LTD., Shanghai (CN)

(72) Inventors: Ziyan WU, Cambridge, MA (US); Meng ZHENG, Cambridge, MA (US); Benjamin PLANCHE, Cambridge, MA (US); Abhishek SHARMA, Cambridge, MA (US); Arun INNANJE, Cambridge, MA (US); Shanhui SUN, Cambridge, MA (US); Terrence CHEN, Cambridge, MA (US)

(73) Assignee: SHANGHAI UNITED IMAGING INTELLIGENCE CO., LTD., Shanghai (CN)

(21) Appl. No.: 17/929,760

(22) Filed: Sep. 6, 2022

(51) Int. Cl. A61B 34/10; A61B 34/30; G16H 20/40; G16H 40/67

(52) U.S. Cl. CPC A61B 34/10; A61B 34/30; G16H 20/40; G16H 40/67; A61B 2090/365

(57) ABSTRACT

A method for surgery planning is provided. The method may include: generating a surgery video displaying a process of a virtual surgery performed by a virtual surgical robot on a virtual patient based on a surgery plan relating to a robotic surgery to be performed on a patient by a surgical robot; transmitting the surgery video to a display component of an XR assembly used to render the surgery video for display to a user; recording one or more actions that the user performed on the virtual surgery based on user input received via an input component of the XR assembly; modifying the surgery plan based on the one or more recorded actions; and causing the surgical robot to implement the robotic surgery on the patient according to the modified surgery plan.

### TECHNICAL FIELD

The disclosure generally relates to the field of surgery planning, and more particularly, relates to systems and methods for surgery planning based on extended reality (XR) techniques.

### BACKGROUND

Preoperative surgery planning is an important part of a surgery, especially, a robotic surgery. With the development of new medical technologies, an application of a robotic surgery system is becoming more and more extensive. A robotic surgery system is usually a complex system that performs a complex surgery, and a planning process of a robotic surgery is also complex. The surgery planning may need to consider a plurality of factors, including a position of a sensor, a position of a surgical robot, a position of a patient, a movement of the surgical robot, a moving path of a surgical instrument, etc. However, most existing technologies of surgery planning may analyze different factors separately, although they have dependencies on each other, which may lead to suboptimal planning, and may introduce and accumulate severe errors. Therefore, it is desirable to provide effective systems and methods for robotic surgery planning.

### SUMMARY

According to an aspect of the present disclosure, a system for surgery planning is provided. The system may include at least one storage device storing a set of instructions, and at least one processor configured to communicate with the at least one storage device. When executing the executable instructions, the at least one processor may be configured to direct the system to perform a plurality of operations.

In some embodiments, the operations may include generating a surgery video displaying a process of a virtual surgery performed by a virtual surgical robot on a virtual patient based on a surgery plan relating to a robotic surgery to be performed on a patient by a surgical robot. The operations may further include transmitting the surgery video to a display component of an XR assembly used to render the surgery video for display to a user, and recording one or more actions that the user performed on the virtual surgery based on user input received via an input component of the XR assembly. The operations may also include modifying the surgery plan based on the one or more recorded actions, and causing the surgical robot to implement the robotic surgery on the patient according to the modified surgery plan.

In some embodiments, the generating a surgery video displaying a process of a virtual surgery may include obtaining first image data of the surgical robot and second image data of the patient; generating the virtual surgical robot representing the surgical robot based on the first image data; generating the virtual patient representing the patient based on the second image data; and generating the surgery video displaying the process of the virtual surgery by animating the virtual surgical robot and the virtual patient based on the surgery plan.

In some embodiments, the surgery video may further display an operation room where the robotic surgery is to be performed, and the generating a surgery video displaying a process of a virtual surgery may include: obtaining third image data of the operation room captured by one or more sensors; and generating a virtual operation room representing the operation room based on the third image data, wherein in the virtual video, the virtual surgical robot and the virtual patient are placed at their respective planning positions specified in the surgery plan in the virtual operation room.

In some embodiments, the recording one or more actions that the user performed on the virtual surgery based on user input received from an input component of the XR assembly may include: determining one or more candidate actions that the user intends to perform on the virtual surgery based on the user input; for each of the one or more candidate actions, updating configurations of the virtual surgical robot and the virtual patient in the surgery video in response to the candidate action; and recording the candidate action as one of the one or more actions in response to determining that the updated configurations satisfy a preset condition.

In some embodiments, the operations further may include: for a target action of the one or more actions predicting possible outcomes of the target action; for each of the possible outcomes, updating configurations of the virtual surgical robot and the virtual patient based on the possible outcome; and recording a response action of the user to the updated configurations based on second user input received via the input component.

In some embodiments, the operations may further include: during the implementation of the robotic surgery: obtaining monitoring information relating to the implementation of the robotic surgery; selecting a target outcome that actually occurs based on the monitoring information from the possible outcomes; and generating a recommendation relating to the response action of the user to the target outcome.

In some embodiments, the operations may further may include during the implementation of the robotic surgery: obtaining monitoring information relating to the implementation of the robotic surgery; determining whether an action to be performed according to the modified surgery plan is risky based on the monitoring information; and in response to determining that the action is risky, generating a notification regarding at least one of the action or a risk associated with the action.

In some embodiments, the XR assembly may be operably connected to the at least one processor via a first network, the surgical robot may be operably connected to the at least one processor via a second network, and at least one of the first network or the second network may include a wireless network.

In some embodiments, the operations may further comprise generating the surgery plan by: generating a virtual operation room representing an operation room where a surgery is to be performed, the virtual operation room including one or more visual representations, the visual representations including the virtual surgical robot and the virtual patient; determining one or more optimization targets relating to the one or more visual representations; and generating the surgery plan by optimizing configurations of the one or more visual representations to meet at least part of the one or more optimization targets.

In some embodiments, the one or more visual representations may further include a virtual surgical instrument that represents a surgical instrument and is operably coupled to the virtual surgical robot, the one or more optimization targets may include an objective function that relates to a deviation of a moving trajectory of the virtual surgical instrument from a planning surgical route, and optimizing configurations of the one or more visual representations to meet at least part of the one or more optimization targets may include obtaining one or more constraints relating to the surgical robot and the surgical instrument; updating configurations of the virtual surgical robot and the virtual surgical instrument based on the one or more constraints to generate a plurality of possible moving trajectories of the virtual surgical instrument; determining a value of the objective function corresponding to each of the plurality of possible moving trajectories; selecting one or more possible moving trajectories whose corresponding values of the objective function satisfy a preset condition from the plurality of possible moving trajectories; and determining a moving trajectory of the surgical instrument during the robotic surgery based on one or more selected possible moving trajectories.

In some embodiments, the operations may further include determining a position of the surgical robot based on the moving trajectory of the surgical instrument.

In some embodiments, the one or more visual representations may further include a virtual sensor representing a sensor, and the one or more optimization targets include an objective function that relates to a coverage rate of a target area in the virtual operation room by a field of view (FOV) of the virtual sensor, and optimizing configurations of the one or more visual representations to meet at least part of the one or more optimization targets may include: updating a configuration of the virtual sensor to determine a plurality of possible sensor positions of the sensor; determining a value of the objective function corresponding to each of the plurality of possible sensor positions; selecting one or more possible sensor positions whose corresponding values of the objective function satisfy a preset condition from the plurality of possible sensor positions; and determining a position of the sensor based on the one or more selected possible sensor positions.

In some embodiments, the one or more visual representations may further include a virtual staffer representing a staffer who assists the robotic surgery and a virtual medical device representing a medical device configured to emit radiation toward the patient during the robotic surgery, the one or more optimization targets include an objective function that relates to a radiation dosage received by the staffer during the robotic surgery, and optimizing configurations of the one or more visual representations to meet at least part of the one or more optimization targets are met may include: updating a configuration of the virtual staffer to determine a plurality of possible staffer positions relative to the virtual medical device; determining a value of the objective function corresponding to each of the plurality of possible staffer positions; selecting one or more possible staffer positions whose corresponding values of the objective function satisfy a preset condition from the plurality of possible staffer positions; and determining a position of the staffer during the robotic surgery based on the one or more selected possible staffer positions.

In some embodiments, the operations may include: generating a virtual operation room representing an operation room where a surgery is to be performed, the virtual operation room including one or more visual representations representing one or more objects located in the operation room during the surgery; determining one or more optimization targets relating to the one or more visual representations; and generating a surgery plan with respect to the surgery by optimizing configurations of the one or more visual representations to meet at least part of the one or more optimization targets.

A method for surgery planning implemented on a computing device having at least one processor and at least one storage device is provided. The method may include: generating a surgery video displaying a process of a virtual surgery performed by a virtual surgical robot on a virtual patient based on a surgery plan relating to a robotic surgery to be performed on a patient by a surgical robot; transmitting the surgery video to a display component of an XR assembly used to render the surgery video for display to a user; recording one or more actions that the user performed on the virtual surgery based on user input received via an input component of the XR assembly; modifying the surgery plan based on the one or more recorded actions; and causing the surgical robot to implement the robotic surgery on the patient according to the modified surgery plan.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary application scenario of a surgery planning system according to some embodiments of the present disclosure;

FIG. 2 is a block diagram illustrating an exemplary surgery planning system according to some embodiments of the present disclosure;

FIG. 3 is a flowchart illustrating an exemplary process for surgery planning and surgery implementing according to some embodiments of the present disclosure;

FIG. 4 is a flowchart illustrating an exemplary process for generating a surgery video according to some embodiments of the present disclosure;

FIG. 5 is a flowchart illustrating an exemplary process for surgery planning and surgery implementation according to some embodiments of the present disclosure;

FIG. 6 is a flowchart illustrating an exemplary process for monitoring the implementation of a robotic surgery according to some embodiments of the present disclosure;

FIG. 7 is a flowchart illustrating an exemplary process for generating a surgery plan for a surgery to be performed on a patient according to some embodiments of the present disclosure;

FIG. 8 is a schematic diagram illustrating exemplary optimization targets according to some embodiments of the present disclosure; and

FIG. 9 is a block diagram illustrating an exemplary processing device according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections, or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or other storage devices. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an erasable programmable read-only memory (EPROM). It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments in the present disclosure. It is to be expressly understood, the operations of the flowchart may be implemented not in order. Conversely, the operations may be implemented in inverted order, or simultaneously. Moreover, one or more other operations may be added to the flowcharts. One or more operations may be removed from the flowcharts.

An aspect of the present disclosure relates to systems and methods for surgery planning using extended reality (XR) techniques. The XR techniques herein may include virtual reality (VR) techniques, augmented reality (AR) techniques, and mixed reality (MR) techniques. The VR techniques may create a computer-generated virtual environment to completely replace a user's view and provide an immersive experience to the user. The AR techniques may enhance the user's view of the real world by overlaying computer-generated content on the real world. The MR techniques may be an extension of the AR techniques that allow real and visual representations to interact in an environment (e.g., a virtual environment). For illustration purposes, the present disclosure primarily describes systems and methods for surgery planning using VR techniques, and this is not intended to be limiting.

In some embodiments, the systems may generate a surgery video displaying a process of a virtual surgery performed by a virtual surgical robot on a virtual patient based on a surgery plan relating to a robotic surgery to be performed on a patient by a surgical robot. The systems may further transmit the surgery video to a display component of an XR assembly used to render the surgery video for display to a user. The systems may also record one or more actions that the user performed on the virtual surgery based on user input received via an input component of the XR assembly, and modify the surgery plan based on the one or more recorded actions. The systems may then cause the surgical robot to implement the robotic surgery on the patient according to the modified surgery plan.

According to some embodiments of the present disclosure, a surgery video displaying the process of a virtual surgery may be generated based on a surgery plan and displayed to a user via an XR assembly. One or more actions performed by the user on the virtual surgery may be recorded for guiding the implementation of the actual robotic surgery. By viewing the surgery video, the user may know the procedure and other implementation details of the robotic surgery to be performed, and find out problems and/or risks in the original surgery plan. By performing the action(s) on the virtual surgery, the user may rehearse the robotic surgery in the virtual surgical environment. By recording the action(s) of the user, the actual robotic surgery may be performed based on the recorded action(s) with no or minimal manual intervention.

In addition, in some embodiments, in response to an action performed by the user, more than one possible update of the configurations of the visual representations in the virtual surgery may be presented to the user to cover different possible outcomes that may occur in the actual robotic surgery. In this way, the user may foresee the different possible outcomes and prepare for the different possible outcomes in advance. Moreover, the surgery planning systems and methods disclosed herein may have high flexibility and convenience because the virtual surgery and the actual robotic surgery may be performed at any location and at any time, respectively, according to an actual need.

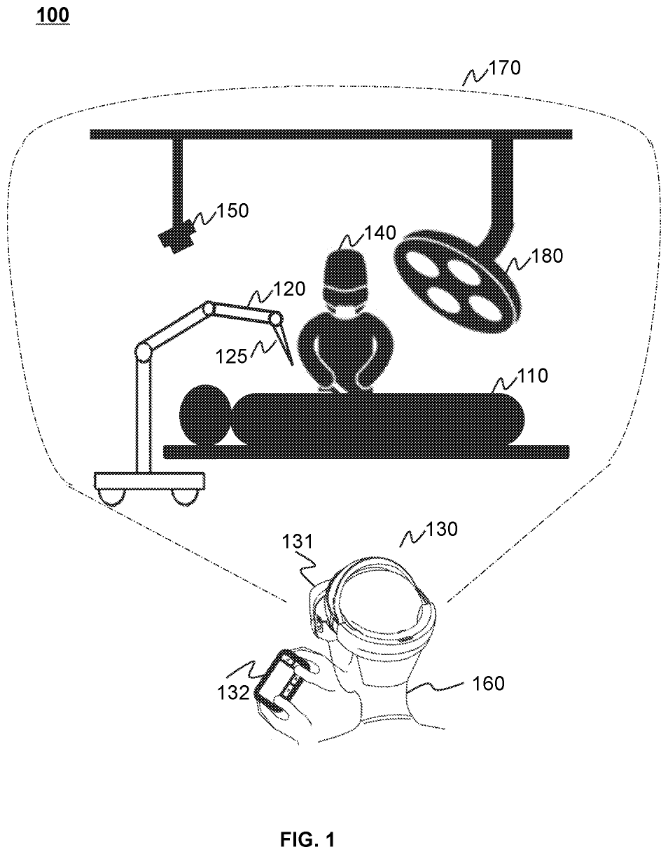

FIG. 1 is a schematic diagram illustrating an exemplary application scenario of a surgery planning system according to some embodiments of the present disclosure.

The surgery planning system may be applied in, for example, surgery planning, surgery training, surgery simulation, surgery rehearsal, surgery programming, etc. The surgery planning may include determining various implementation details of a surgery, including a surgery procedure, a position of an object (e.g., a sensor, a surgical robot, a staffer, a patient, etc.) in an operation room, a surgical goal, a moving path of a surgical instrument, a moving mode of the surgical robot, or the like, or any combination thereof. The surgery rehearsal refers to providing a user with a simulation environment for exercising the surgery procedure, so that the user can practice surgery operations, be familiar with the surgery procedure, and prepare for the actual surgery. The surgery programming refers to recording the user's behavior during the surgery rehearsal and performing an actual surgery based on the recorded behavior, which can reduce manual intervention during the actual surgery.

A surgery in the present disclosure may include a robotic surgery and/or an artificial surgery. As used herein, if a surgical robot participates in a surgery of a subject, the surgery may be deemed as a robotic surgery. For example, actions to be performed by the surgical robot may be previously determined and programmed as instructions, and a processor of the surgical robot may automatically control the surgical robot to perform the surgery on the subject according to the programmed instructions without any user intervention. As another example, the surgical robot may perform the robotic surgery under the supervision and control of a user. Merely by way of example, the user may remotely control the surgical robot by manipulating control component(s) (e.g., a foot-pedal, keys, a touch screen, a joystick) of a console. As yet another example, the surgical robot may perform the surgery based on both the programmed instructions and the user intervention. If the surgery is performed by humans without any assistance from a surgical robot, the surgery may be deemed as an artificial surgery.

A subject who receives a surgery may include an animal, a patient (or a portion thereof), etc. For illustration purposes, the present disclosure describes a process of surgery planning by taking a robotic surgery performed on a patient as an example. It should be understood that the systems and methods for surgery planning in the present disclosure may also be used for other subjects and/or other types of surgeries.

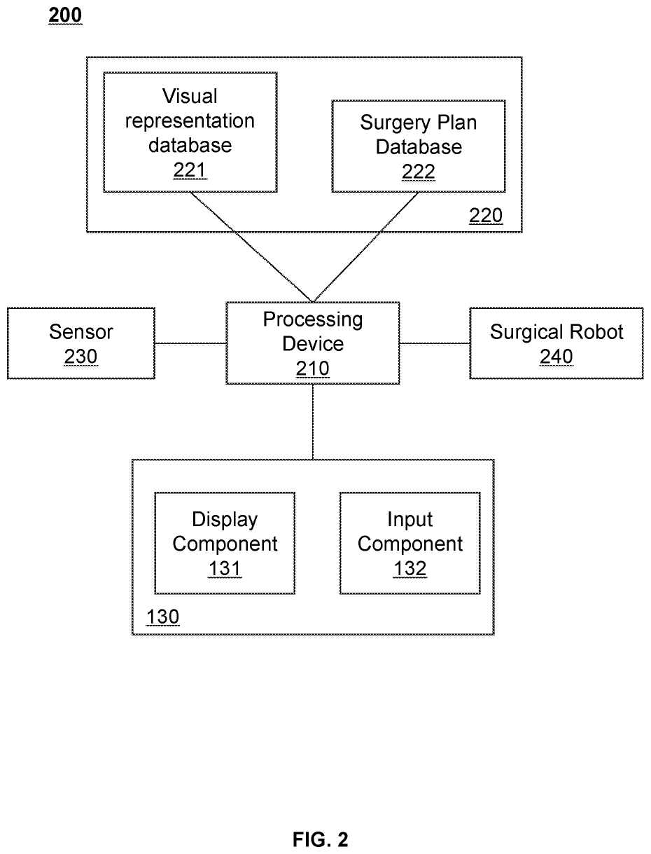

In some embodiments, as shown in FIG. 1, the application scenario 100 of the surgery planning system may include a user 160, an XR assembly 130, and virtual content 170. The user 160 may interact with the virtual content 170 through the XR assembly 130. For example, the XR assembly 130 may include a display component 131 and an input component 132. The display component 131 may be configured to display the virtual content 170 (e.g., a virtual operation room, a surgery video displaying a process of a virtual surgery) to the user 160. The input component 132 may be configured to receive user inputs for manipulating the virtual content 170 inputted by the user 160. More descriptions regarding the XR assembly 130 may be found in FIG. 2.

The virtual content 170 may present a virtual surgery environment including a plurality of visual representations, such as a virtual operation room, a virtual patient 110, a virtual surgical robot 120, a virtual surgical instrument 125, a virtual staffer 140, a virtual sensor 150, a virtual lighting device 180, a virtual display (not shown in FIG. 1), a virtual console (not shown in FIG. 1), or the like, or any combination thereof. The virtual surgical instrument 125 may be installed at an end of a virtual robotic arm of the virtual surgical robot 120. The virtual surgical robot 120 may perform a virtual surgery on the virtual patient 110 by manipulating the virtual surgical instrument 125.

Each visual representation in the virtual content 170 may represent a real object in a real operation room. For example, a virtual operation room may represent an operation room where a robotic surgery is to be performed. The virtual patient 110 may represent a patient who needs to receive the robotic surgery. The virtual surgical robot 120 may represent a surgical robot that performs the robotic surgery. The virtual surgical instrument 125 may represent a surgical instrument, such as a scalpel, a surgical scissor, a surgical hemostat, a surgical pull hook, a surgical suture needle, an endoscope, etc. The virtual staffer 140 may represent a staffer participating in the robotic surgery, such as a nurse, an anesthesiologist, a surgeon, etc. The virtual sensor 150 may represent a sensor in the operation room. It can be understood that a position of a visual representation in the virtual surgery environment may be consistent with a real position of the real object that the visual representation represents in the real operation room. An appearance and an internal structure of a visual representation may be consistent with the real object it represents.

In some embodiments, a visual representation in the virtual content 170 may be represented as a point cloud model, a 3D mesh model, a CAD model, a 3D model reconstructed from image data (e.g., optical image data captured by a sensor, medial image data captured by a medical scan), a mathematical model (e.g., a mathematical model of a radiation source), a kinematic model (e.g., a robotic kinematic model), or the like, or any combination thereof.

In some embodiments, an initial configuration of a visual representation may be determined and/or updated based on user input. In some embodiments, the initial configuration of the visual representation may be determined and/or updated based on engineering data, such as a CAD model, a drawing, or a floorplan. In some embodiments, the initial configuration of the visual representation may be determined and/or updated based on data (e.g., an image, a video) captured by a sensor in the operation room and/or data collected by a medical scan. In some embodiments, the initial configuration of the visual representation may be determined and/or updated based on physics laws, mathematical models, etc.

In some embodiments, the virtual surgery environment may interact with the real surgery environment. For example, the user may rehearse the robotic surgery to be performed in the real surgery environment through the virtual surgery environment. As another example, the user may modify a surgery plan of the robotic surgery to be performed in the real surgery environment based on his/her interaction with the virtual surgery environment.

FIG. 2 is a block diagram illustrating an exemplary surgery planning system according to some embodiments of the present disclosure. As shown in FIG. 2, a surgery planning system 200 may include a processing device 210, a storage device 220, a sensor 230, a surgical robot 240, an XR assembly 130, etc.

The XR assembly 130 may include a device that allows a user to be engaged in an extended reality experience. For example, XR assembly 130 may include a VR assembly, an AR assembly, an MR assembly, or the like, or any combination thereof. In some embodiments, the XR assembly 130 may include an XR helmet, XR glasses, an XR patch, a stereoscopic headset, or the like, or any combination thereof. For example, the XR assembly 130 may include a Google Glass™, an Oculus Rift™, a Gear VR™, etc. Specifically, the XR assembly 130 may include a display component 131 on which virtual content may be rendered and/or displayed. The user may view the virtual content (e.g., a surgery video, a virtual surgery environment) via the display component 131.

In some embodiments, the user may interact with the virtual content via the display component 131. For example, when the user wears the display component 131, head motion and/or a gaze direction of the user may be tracked so that the virtual content may be rendered responsive to changes in the user's position and/or orientation to provide an immersive extended reality experience that reflects changes to the user's perspective.

In some embodiments, the XR assembly 130 may further include an input component 132. The input component 132 may enable user interactions between a user and the virtual content (e.g., the virtual surgery environment) displayed by the display component 131. For example, the input component 132 may include a touch sensor, a microphone, etc., configured to receive user input, which may be provided to the XR assembly 130 and used to control the virtual world by varying the visual content rendered on the display component 131. In some embodiments, the user input received by the input component 132 may include, for example, touch, voice input, and/or hand gesture input, and may be sensed via any suitable sensing technology (e.g., capacitive, resistive, acoustic, optical). In some embodiments, the input component 132 may include a handle, a glove, a stylus, a game console, etc.

In some embodiments, the display component 131 (or the processing device 210) may track the input component 132. In some embodiments, tracking information acquired from the tracking of the input component 132 may be processed for rendering a visual representation. The processing of the tracking information and/or the rendering may be performed by the display component 131, a processing device (e.g., the processing device 210) operably connected via, e.g., a wired or wireless network, to the display component 131, or the like, or a combination thereof. The visual representation may include a representation of the input component 132 (e.g., an image of the user's hand, or fingers). The visual representation may be rendered in a 3D location in the extended reality experience that corresponds to the real-world location of the input component 132. For example, one or more second sensors may be used for tracking the input component 132. The display component 131 may receive signal(s) collected by the one or more second sensors from the input component 132 via a wired or wireless network. The signal(s) may include any suitable information enabling the tracking of the input component 132, such as an output from one or more inertial measurement units (e.g., an accelerometer, a gyroscope, a magnetometer) in the input component 132, a global positioning system (GPS) sensor in the input component 132, etc. The signal(s) may indicate the position (e.g., in a form of three-dimensional coordinates) and/or orientation (e.g., in a form of three-dimensional rotational coordinates) of the input component 132. In some embodiments, the second sensor(s) may include one or more optical sensors for tracking the input component 132. For example, the second sensor(s) may employ a visible light and/or depth camera to locate the input component 132.

The surgical robot 240 may be a mechanical system configured to perform or assist a surgery. For example, the surgical robot 240 may have the same or similar structure as the virtual surgical robot 120 shown in FIG. 1. The surgical robot 240 may include a robotic arm, a motion component, a motor, etc. The robotic arm may be a mechanical arm configured to control the movement of a surgical instrument. For example, the surgical instrument may be coupled to the robotic arm (e.g., the end of the robotic arm). The robotic arm may include a plurality of links connected by one or more joints, and the combination of the links and the joint(s) allows the surgical instrument to have rotational motion and/or translational disposition. The motion component may drive the robot to move on the ground and/or change the height of the robotic arm, etc. For example, the motion component may include a pulley, a lifting ladder, etc.

In some embodiments, the surgical instrument controlled by the surgical robot may include, for example, a scalpel, a surgical scissor, a clamp, an endoscope, a puncture needle, etc. In some embodiments, the surgical robot 240 may be mounted on an operation platform (e.g., a table, a bed, etc.), a cart, a ceiling, a sidewall or any other suitable supporting plane.

The processing device 210 may process data and/or information obtained from the storage device 220, the XR assembly 130, and/or any other components. In some embodiments, the processing device 210 may host a simulated virtual world or a metaverse for the XR assembly 130. In some embodiments, the processing device 210 may host a virtual surgery environment. For example, the processing device 210 may generate, based on surgery-related data collected by the sensor 230, a virtual operation room including a virtual surgical robot and a virtual patient. As another example, the processing device 210 may generate a surgery video displaying the process of a virtual surgery by animating the virtual surgical robot and the virtual patient based on a surgery plan. As yet another example, the processing device 210 may record one or more actions that a user performed on the virtual surgery, and update configurations of the virtual surgical robot and/or the virtual patient in the surgery video based on the recorded action(s).

In some embodiments, the processing device 210 may generate a visual representation of an object by processing data of the object. The data of the object may include point cloud data, depth data, time of flight (TOF) data, three-dimensional image data, etc., or a combination thereof. In some embodiments, the processing device 210 may generate metadata relating to the visual representation, such as the equipment type, the location, and the orientation of the visual representation, so that the XR assembly 130 can render the visual representation into the extended reality environment based on the metadata. In some embodiments, the visual representation(s) generated by the processing device 210 may be stored in the visual representation database 221. In some embodiments, the processing device 210 may retrieve a visual representation stored in the visual representation database 221, and process the retrieved visual representation. For example, the processing device 210 may animate the received visual representation to generate a surgery video. As another example, the processing device 210 may receive and utilize different combinations of visual representations to generate a particular virtual surgery environment.

In some embodiments, the processing device 210 may embed one or more kinematic algorithms (e.g., one or more kinematic constraints) in the virtual surgery environment. The kinematic algorithm(s) may be constructed based on the behaviors of an object, such as a surgical robot, a patient, etc., in the real surgery environment, and may be configured to describe the behavior of a visual representation corresponding to the object in the virtual surgery environment. Merely by way of example, one or more kinematic constraints may be loaded as a “wrapper” over a virtual robotic arm to define the kinematic behavior of the virtual robotic arm, for example, define how the virtual robotic arm responds to user interactions (e.g., moving the virtual robotic arm by selection and manipulation of a touchpoint on the virtual robotic arm), or how the virtual robotic arm operates in a control mode selected or specified by the surgery plan. By embedding in the virtual surgery environment the one or more kinematic algorithms that accurately describe behaviors of the object in the actual surgery environment, the visual representation of the object in the virtual surgery environment may operate and function accurately and realistically.

In some embodiments, the processing device 210 may be a computer, a user console, a single server, or a server group, etc. The server group can be centralized or distributed. For example, a specified area of the metaverse may be simulated by a single server. In some embodiments, the processing device 210 may include a plurality of simulation servers dedicated to physics simulation in order to manage interactions and handle collisions between characters and objects in the metaverse. Although one processing device 210 is described in FIG. 2, computing functions relating to the surgery planning system 200 described in the present disclosure may be implemented in a distributed fashion by a group of similar platforms to spread the processing load of the surgery planning system 200.

In some embodiments, the processing device 210 may be local to or remote from the surgery planning system 200. For example, the processing device 210 may access information and/or data from the storage device 220 via the network. As another example, the processing device 210 may be directly connected to the storage device 220 to access information and/or data. In some embodiments, the processing device 210 may be implemented on a cloud platform. For example, the cloud platform may include a private cloud, a public cloud, a hybrid cloud, a community cloud, a distributed cloud, and inter-cloud, a multi-cloud, or the like, or a combination thereof. In some embodiments, the processing device 210 may be implemented by a computing device having a processor, a storage, an input/output (I/O), a communication port, etc. In some embodiments, the processing device 210 may be implemented on a processing circuit (e.g., a processor, a CPU) of the XR assembly 130.

The storage device 220 may be configured to store data and/or instructions. In some embodiments, the storage device 220 may include a random access memory (RAM), a read-only memory (ROM), a mass storage device, a removable storage device, a volatile read-and-write memory, or the like, or any combination thereof. Exemplary mass storage devices may include a magnetic disk, an optical disk, a solid-state drive, etc. In some embodiments, the storage device 220 may be implemented on a cloud platform. In some embodiments, the storage device 220 may be integrated into or included in one or more other components (e.g., the XR assembly 130, the surgical robot 240, the processing device 210). In some embodiments, the storage device 220 may store instructions for the processing device 210 to execute to perform the surgery planning methods disclosed herein.

In some embodiments, the storage device 220 may include a visual representation database 221 and/or a surgery plan database 222.

The visual representation database 221 may be configured to store data relating to objects and characters in the virtual surgery environment. The data stored in the visual representation database 221 may include object shapes, visual representation shapes and appearances, audio clips, metaverse related scripts, and other metaverse related objects. In some embodiments, the visual representation database 221 may store one or more configuration files describing the visual representation(s) of the virtual surgery environment. For example, the visual representation database 221 may store files describing different kinds of operation rooms (e.g., varying in room shape or room dimensions), operating tables (e.g., varying in size, height, surfaces, material construction, etc.), robotic arms (e.g., varying in the design of arm links and joints, number and arrangement thereof, number and location of virtual touchpoints on the arm, etc.), patient types (e.g., varying in sex, age, weight, height, girth, etc.) and/or medical personnel (e.g., generic graphical representations of people, graphical representations of a specific staffer, etc.). In some embodiments, a configuration file in Unified Robot Description Format (URDF) may be used to store data of a robotic arm.

The surgery plan database 222 may store surgery plan information and/or patient information. The surgery plan information may include any information related to the implementation of a surgery, such as a surgery procedure, a position of an object in an operation room (such as a sensor, a surgical robot, staffer, a patient, etc.), a surgical goal, a moving path of a surgical instrument, a moving mode of a surgical robot, etc. The patient information may include, for example, patient imaging data (e.g., X-ray, MRI, CT, ultrasound, etc.), medical histories, and/or patient profile information (e.g., age, weight, height, etc.).

In some embodiments, the processing device 210 may acquire surgery planning information and/or patient information from the surgery plan database 222 and utilize the acquired information in the generation of the virtual surgery environment. For example, a representation of a patient's anatomy may be generated and overlaid over a portion of a user's field of view of the virtual surgery environment (e.g., overlaying an ultrasound image showing the patient's tissue over the virtual patient's tissue), which may be useful, for example, for determining a desirable arrangement of robotic arms around the patient.

The sensor 230 may be configured to collect information relating to components, for example, the surgical robot, the patient, etc., in the actual surgery environment. For example, the sensor 230 may be configured to detect a position, an orientation, a velocity, etc., of a component of the surgical robot 240. As another example, the sensor 230 may be configured to detect a position, an orientation, etc., of a surgical instrument mounted on the surgical robot 240. In some embodiments, the sensor 230 may include a camera, a speed sensor, a position sensor, an angle sensor, or the like, or any combination thereof. Exemplary cameras may include an RGB camera, a depth camera, a structured light camera, a laser camera, or a time-of-flight camera, an infrared camera, or the like, or any combination thereof. In some embodiments, various sensors may be used to collect various information relating to multiple components in the actual surgery environment.

In some embodiments, the surgery planning system 200 may further include an audio device (not shown) configured to provide an audio signal to the user. For example, the audio device (e.g., a speaker) may play a sound (e.g., a notification sound regarding possible collision in the virtual or actual surgery environment). In some embodiments, the audio device may include an electromagnetic speaker (e.g., a moving-coil speaker, a moving-iron speaker, etc.), a piezoelectric speaker, an electrostatic speaker (e.g., a condenser speaker), or the like, or any combination thereof. In some embodiments, the audio device may be integrated into the XR assembly 130. In some embodiments, the XR assembly 130 may include two audio devices respectively located on the left and right sides of the XR assembly 130 to provide audio signals to the user's left and right ears.

It should be noted that the above description of the surgery planning system 200 and its application scenario is merely provided for the purposes of illustration, and not intended to limit the scope of the present disclosure. For persons having ordinary skills in the art, multiple variations and modifications may be made under the teachings of the present disclosure. For example, the assembly and/or function of the surgery planning system 200 may be varied or changed according to specific implementation scenarios. In some embodiments, the surgery planning system 200 may include one or more additional components (e.g., a storage device, a network, etc.) and/or one or more components of the surgery planning system 200 described above may be omitted. Additionally or alternatively, two or more components of the surgery planning system 200 may be integrated into a single component. A component of the surgery planning system 200 may be implemented on two or more sub-components. In some embodiments, the visual representation database 221 and/or the surgery plan database 222 may be external databases connected to the surgery planning system 200. As another example, the visual representation database 221 and the surgery plan database 222 may be integrated into one database.

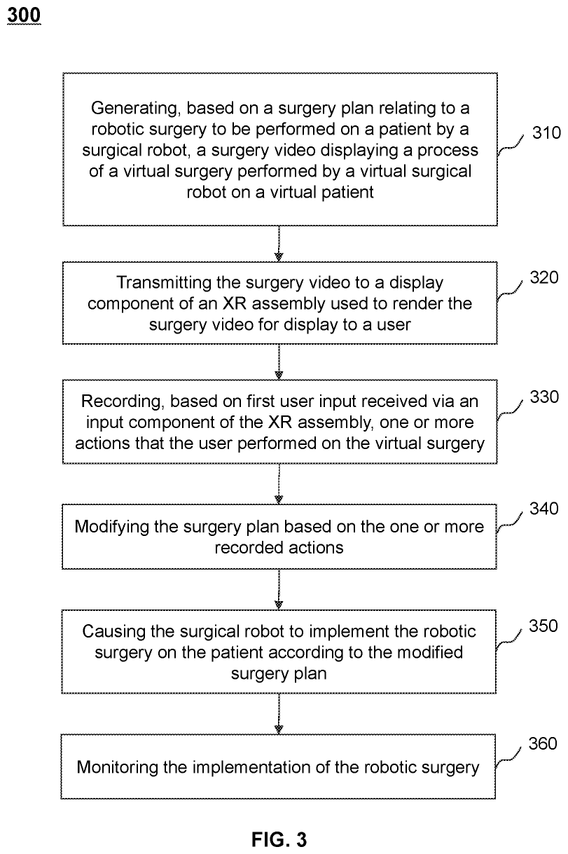

FIG. 3 is a flowchart illustrating an exemplary process for surgery planning and surgery implementing according to some embodiments of the present disclosure. In some embodiments, a process 300 may be implemented as a set of instructions (e.g., an application) stored in a storage device. The processing device 210 may execute the set of instructions, and when executing the instructions, the processing device 210 may be configured to perform the process 300. The operations of the illustrated process 300 presented below are intended to be illustrative. In some embodiments, the process 300 may be accomplished with one or more additional operations not described, and/or without one or more of the operations discussed. Additionally, the order of the operations of the process 300 illustrated in FIG. 3 and described below is not intended to be limiting.

In 310, the processing device 210 may generate, based on a surgery plan relating to a robotic surgery to be performed on a patient by a surgical robot, a surgery video displaying a process of a virtual surgery performed by a virtual surgical robot on a virtual patient. In some embodiments, operation 310 may be performed by a generation module 910 of the processing device 210.

The robotic surgery may refer to a surgery that a surgical robot participates in and performs at least a portion thereof. The robotic surgery may include a surgery completely performed by a surgical robot, a surgery performed by the surgical robot and a staffer together, etc.

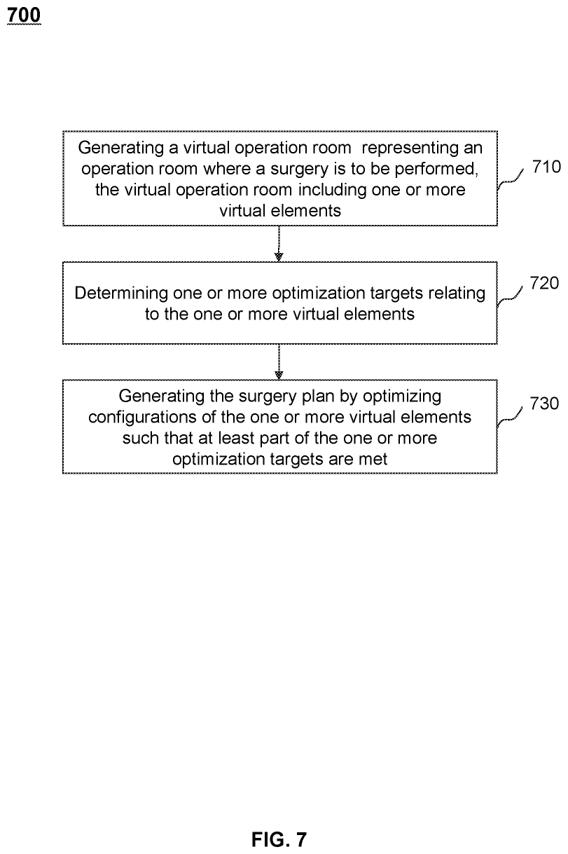

The surgery plan may define how the robotic surgery is performed on a patient. The surgery plan may include various information relating to the robotic surgery. For example, the surgery plan may include an implementation procedure of the robotic surgery, planning positions of a plurality of objects (such as the surgical robot, the patient, staffer, a sensor, etc.) in an operation room, a moving trajectory of the surgical robot, a surgical path of a surgical instrument, a goal that the surgery needs to achieve, an organ or tissue of the patient that needs to be avoided during the movement of the surgical instrument, etc. In some embodiments, the surgery plan may be generated by performing process 700 as described in connection with FIG. 7.

The surgery video may display a procedure of the virtual surgery performed by the virtual surgical robot on the virtual patient. In some embodiments, the surgery video may be a VR video that includes a virtual surgery environment, which may completely replace a user's view and provide an immersive experience to the user. In some embodiments, the surgery video may be an AR video that includes a plurality of visual representations, which may be overlaid on the real surgery environment, or a virtual representation thereof. In some embodiments, the surgery video may be an MR video that includes the plurality of visual representations of the AR video, and the visual representations may interact with the real surgery environment.

In some embodiments, the surgery video may be a VR video including a virtual operation room. The virtual operation room may refer to a virtual world corresponding to a real operation room. The virtual operation room may include a plurality of visual representations corresponding to a plurality of objects in the real operation room, such as a virtual surgical robot, a virtual patient, a virtual staffer, a virtual surgical instrument, a virtual sensor, a virtual imaging device, etc. In the surgery video, the visual representations may operate as defined in the surgery plan. For example, the virtual surgical robot may be located at its planned position, and a virtual robotic arm of the virtual surgical robot may move so that the virtual surgical instrument coupled to an end of the virtual robotic arm moves along its planned moving trajectory. More descriptions regarding the virtual operation room and the visual representations thereof may be found elsewhere in the present disclosure. See, e.g., FIGS. 1-2 and relevant descriptions thereof.

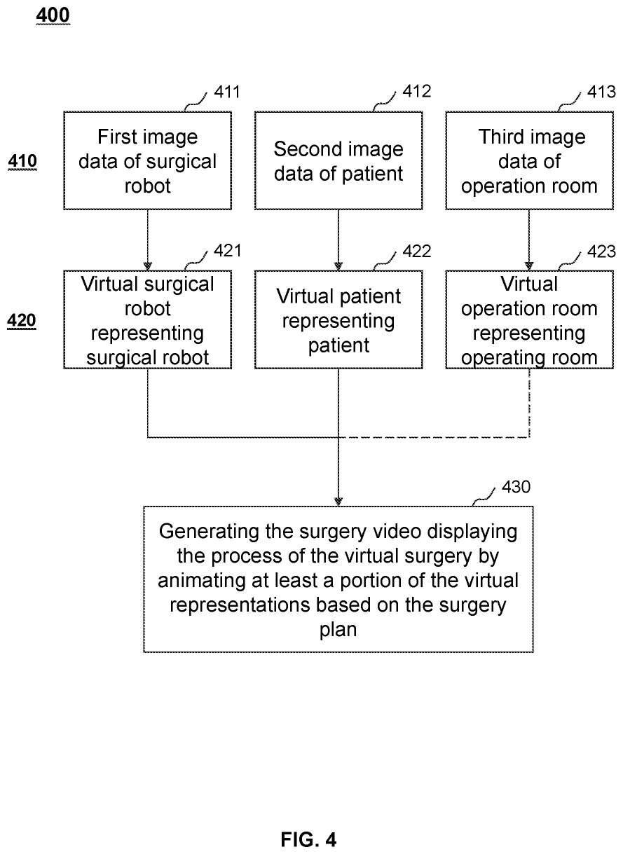

In some embodiments, the processing device 210 may obtain first image data of the surgical robot and second image data of the patient. The processing device 210 may generate the virtual surgical robot representing the surgical robot based on the first image data, and generate the virtual patient representing the patient based on the second image data. The processing device 210 may generate the surgery video displaying the process of the virtual surgery by animating the virtual surgical robot and the virtual patient based on the surgery plan. More descriptions regarding the generation of the surgery video may be found elsewhere in the present disclosure. See, e.g., FIG. 4 and relevant descriptions thereof.

In 320, the processing device 210 may transmit the surgery video to a display component of an XR assembly used to render the surgery video for display to a user. In some embodiments, operation 320 may be performed by a transmission module 920 of the processing device 210.

In some embodiments, the processing device 210 may transmit the surgery video to the display component of the XR assemble via a network. The XR assembly may be used to render the surgery video for display to the user. In some embodiments, the visual rendering operation may include a visual transformation, a color operation, a lighting/illuminating operation, a texture mapping operation, an animation effect operation, or the like, or a combination thereof.

In some embodiments, the surgery video may be rendered based on a specific perspective. Merely by way of example, the surgery video may be rendered based on the virtual surgical robot's perspective or the virtual staffer's perspective in the virtual surgery environment. In this way, the user may view the surgery process from the virtual surgical robot's perspective or the virtual staffer's perspective for a better control and/or monitoring of the virtual surgery. In some embodiments, if the surgery video is a VR video, the display component may immerse the user within the virtual surgery environment, and the user may not see a physical environment where the user is actually located at the time. If the surgery video is an AR video, the user may need to view the surgery video in the actual operation room, and the display component may overlay the surgery video on the actual operation room.

In some embodiments, if the display component of the XR assembly includes a first display component corresponding to the left eyes and a second display component corresponding to the right eyes, the processing device 210 may render a first video corresponding to a first eye view and a second video corresponding to a second eye view based on the surgery video. The processing device 210 may direct the first display component to display the first video and the second display component to display the second video to the user. For example, the first video may correspond to the left eye view and be displayed by the first display component wearing on the left eye of the user, and the second video may correspond to the right eye view and be displayed by the second display component wearing on the right eye of the user.

In 330, the processing device 210 may record, based on first user input received via an input component of the XR assembly, one or more actions that the user performed on the virtual surgery. In some embodiments, operation 330 may be performed by a recording module 930 of the processing device 210.

When the XR assembly displays the surgery video to the user, the user may perform one or more actions on the virtual surgery by inputting the first user input into the input component of the XR assembly. Exemplary action(s) performed by the user on the virtual surgery may include replacing the virtual surgical instrument, changing a position of an insertion point where the virtual surgical instrument is inserted into the virtual patient, changing a moving direction of the virtual surgical instrument, changing a position of the virtual surgical robot, deforming the virtual robotic arm of the virtual surgical robot, changing the surgical procedure, or the like, or any combination thereof.

In some embodiments, the user may input the first user input through the input component by typing, speaking, touching, drawing, etc. Merely by way of example, the XR assembly may display a virtual representation representing to the user, such as a virtual hand or a virtual character. The user may manipulate the virtual hand or the virtual character to drag the virtual robotic arm of the virtual surgical robot so as to adjust the position and/or orientation of the virtual robotic arm.

In some embodiments, the processing device 210 may record every action performed by the user on the virtual surgery. In some embodiments, the processing device 210 may record some actions performed by the user on the virtual surgery. For example, the processing device 210 may only record action(s) that satisfy certain condition(s). Merely by way of example, the processing device 210 may determine, based on the first user input, one or more candidate actions that the user intends to perform on the virtual surgery. The candidate action(s) may include all action(s) that the user performs on the virtual surgery. For each of the one or more candidate actions, configurations of the virtual surgical robot and the virtual patient in the surgery video may be updated in response to the candidate action. The processing device 210 may record the candidate action as one of the one or more actions if the updated configurations satisfy a preset condition.

The updating the configuration(s) of the virtual surgical robot may include, for example, updating the position of the virtual surgical robot, updating the position and/or the orientation of the virtual robotic arm of the virtual surgical robot, updating the virtual surgical instrument coupled to the virtual robotic arm, or the like, or any combination thereof. For example, a first user input may include an instruction for replacing a first surgical instrument with a second surgical instrument, the processing device 210 may replace a first visual representation representing the first surgical instrument coupled to the virtual robotic arm with a second visual representation representing the second surgical instrument (e.g., obtained from the visual representation database 221). As another example, based on the anatomy of the patient, or a portion thereof, and/or prior knowledge of human anatomy, the processing device 210 may update the configuration of the virtual patient (e.g., to simulate the surgery effect) if the moving trajectory of the virtual surgical instrument is adjusted. The prior knowledge of human anatomy may include anatomy information of a general population or a group of persons sharing a characteristic. The general population or the group may include the patient or not include the patient. The group of persons may share a characteristic relevant to the surgery or the virtual representation of the surgery at issue. For instance, the patient has a lesion on the left lung that needs to be removed in a robotic surgery, a characteristic of the group of persons may include age, gender, height, weight, girth, the location of a lesion, the dimension of the lesion, progression of the lesion, etc., or a combination thereof, of a person of the group.

In some embodiments, a candidate action may cause an update of the configuration of one or more other visual representations in the virtual operation room. Merely by way of example, an orientation of a virtual camera may be updated in response to a candidate action. In some embodiments, the configuration updating of a visual representation may be performed based on one or more kinematic algorithms embedded in the virtual surgery environment. As described in connection with FIG. 1, the kinematic algorithm(s) may be constructed based on the behaviors of objects in the actual surgery environment and define how a visual representation responds to user interactions.

The preset condition may include various types of preset conditions. For example, the preset condition may be that an updated virtual surgical robot will not collide with other virtual components, the virtual surgical instrument will not deviate from a planned surgical path (e.g., a deviation of the updated moving trajectory of the virtual surgical instrument from its planned moving trajectory is below a threshold), the updated configurations of the virtual surgical robot and the virtual patient are confirmed by the user, etc.

In some embodiments, the preset condition may include that the updated configurations of the virtual surgical robot and the virtual patient are confirmed by the user. For example, the user and/or the processing device 210 may determine whether the candidate action causes a desired result based on the updated configurations of the virtual surgical robot and the virtual patient. If the user and/or the processing device 210 determines that the candidate action causes a desired result, the processing device 210 may record the candidate action as one of the one or more actions. If the user and/or the processing device 210 determines that the candidate action doesn't cause a desired result, the processing device 210 may not record the candidate action.

In some embodiments of the present disclosure, the user may test possible effects of an operation through the virtual surgery, and then determine whether the candidate action should be designated as an action (or a candidate action) to be performed during the actual surgery. In other words, action(s) executed in the actual surgery can be tested and/or verified by the user before the actual surgery via the virtual surgery, thereby improving the accuracy and/or efficacy, and/or reducing risks of the surgery.

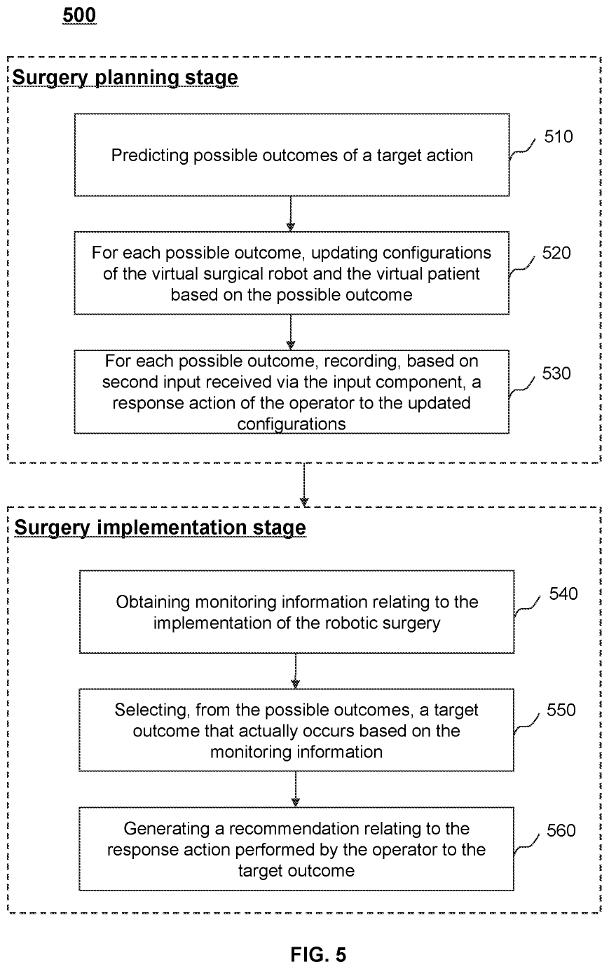

On some occasions, a target action performed by the user on the virtual surgery may cause different possible outcomes. Therefore, for the target action the user performed, more than one possible update of configurations of one or more visual representations (e.g., the virtual surgical robot and/or the virtual patient) in the virtual surgery environment may be presented to the user to cover different possible outcomes that can occur in the actual surgery. A response action of the user toward the updated configurations corresponding to each possible outcome may be recorded by the processing device 210 so that depending on the development in the actual surgery, appropriate actions can be executed accordingly. More descriptions regarding the target action may be found elsewhere in the present disclosure. See, e.g., FIG. 5 and relevant descriptions thereof.

In 340, the processing device 210 may modify the surgery plan based on the one or more recorded actions. In some embodiments, operation 340 may be performed by a modification module 940 of the processing device 210.

For example, the processing device 210 may modify the surgery procedure based on the one or more recorded actions. The original surgery procedure may be “anesthesia, disinfection, mass drainage, and suture,” and the surgery procedure may be modified as “anesthesia, disinfection, mass drainage, mass excision, and suture.” As another example, the processing device 210 may adjust planned positions of the surgical robot, the patient, the staffer, etc., as defined in the surgery plan. The original planned positions may be that “the patient is in a head-high-right-high position, and the surgical robot and the staffer are on the right side of the patient,” and the planned positions may be modified as “the patient is in a horizontal position, and the surgical robot and the staffer are on both sides of the patient.” As yet another example, the processing device 210 may adjust the moving trajectory of the surgical instrument defined in the surgery plan. The original moving trajectory of the surgical instrument may be that “the insertion angle of the surgical instrument is 90°,” and the moving trajectory may be modified as “the insertion angle of the surgical instrument is 45°.”

In 350, the processing device 210 may cause the surgical robot to implement the robotic surgery on the patient according to the modified surgery plan. In some embodiments, operation 350 may be performed by a control module 950 of the processing device 210.

In some embodiments, the processing device 210 may cause the surgical robot to implement the robotic surgery on the patient according to the modified surgery plan. For example, the processing device 210 may cause the surgical robot to move to a planned position of the surgical robot defined in the modified surgery plan, and control the robotic arm of the surgical robot to move according to a moving trajectory of the robotic arm defined in the modified surgery plan, etc.

In some embodiments, the modified surgery plan may describe one or more operations that need to be manually performed by a staffer who assists the robotic surgery, for example, reposition of the surgical instrument used by the surgical robot, a hemostatic operation, an anesthesia operation, or the like, or any combination thereof. In the implementation of the robotic surgery, one or more notification messages (e.g., by way of audio messages, text messages) regarding the operation(s) may be generated and displayed to the staffer to remind he/she to perform the operation(s) at suitable time.

In some embodiments, the user who participates in the virtual surgery and the staffer who participates in the actual robotic surgery may be the same person or different persons. In some embodiments, the user may be located at a first location different from (e.g., remote from) a second location where the actual robotic surgery is performed. The XR assembly may be operably connected to the processing device 210 via a first network, the surgical robot may be operably connected to the processing device 210 via a second network, and at least one of the first network or the second network is a wireless network. For example, the user may be located in city A, and wear the XR assembly that is operably connected to the processing device 210 via a first wireless network. The surgical robot may be located at city B and operably connected to the processing device 210 via a second wireless network. The processing device 210 may be implemented on a cloud platform. In this way, the user (e.g., an expert) may remotely perform the virtual surgery in city A, while the actual robotic surgery may be performed in city B.

In some embodiments, the user may perform the virtual surgery earlier than the actual robotic surgery. For example, the user may perform, based on an original surgery plan, the virtual surgery in advance in through the surgery planning system, and then a modified surgery plan may be generated. In some embodiments, the modified surgery may include a modification that may improve or optimize the surgery in some aspect (e.g., improving the execution of the robotic surgery, reducing the risk of a medical accident, reducing the risk of a collision among different devices involved in the robotic surgery, etc., or a combination thereof), a candidate action for the surgical, or a portion thereof, that may be invoked during the surgery if a condition unexpected or different from the original surgery plan occurs, or the like, or a combination thereof. Examples of such conditions may include a change in size, composition, and/or location of a lesion to be treated in the patient, a medical/health condition of the patient that has newly developed between the time when the original surgery plan was created and the time when the surgery is actually performed or during the period when the surgery is performed (e.g., a hemorrhage that has occurred unexpectedly, anatomical information unknown at the time when the original surgery plan was created, or the like, or a combination thereof. The surgical robot and other users may perform the robotic surgery on the patient according to the modified surgery plan. The virtual surgery and the actual robotic surgery may be performed at any location and at any time according to an actual need.

In 360, the processing device 210 may monitor the implementation of the robotic surgery. In some embodiments, operation 360 may be performed by the control module 950 of the processing device 210.

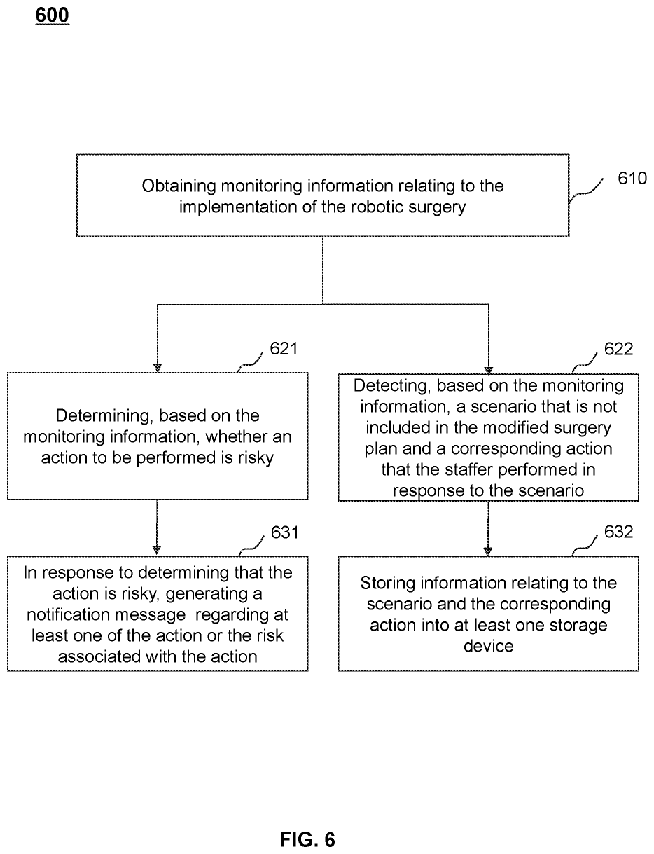

In some embodiments, the processing device 210 may obtain monitoring information relating to the implementation of the robotic surgery, and monitor the implementation of the robotic surgery based on the monitoring information. For example, the processing device 210 may determine, based on the monitoring information, whether an action to be performed is risky. As another example, the processing device 210 may perform collision detection based on the monitoring information to detect collisions between different objects (e.g., the surgical robot and a patient table) in the operation room, and provide a notification (e.g., setting off an alarm) notifying the staffer of the occurrence, or a risk of the occurrence, of the detected collision. As yet another example, based on the monitoring information, the processing device 210 may detect positions of the robotic arm, the surgical instrument, etc., and compare the detected positions with their corresponding expected positions as defined in the modified surgery plan, such that deviations from the surgery plan may be detected and trigger the staffer to perform adjustment to avoid collisions or risks. More descriptions regarding the monitoring of the implementation of the robotic surgery may be found elsewhere in the present disclosure. See, e.g., FIG. 6 and relevant descriptions thereof.

According to some embodiments of the present disclosure, a surgery video displaying the process of a virtual surgery may be generated based on a surgery plan and displayed to a user via an XR assembly. One or more actions performed by the user on the virtual surgery may be recorded for guiding the implementation of the actual robotic surgery. By viewing the surgery video, the user may know the procedure and other implementation details of the robotic surgery to be performed, and find out problems and/or risks in the original surgery plan. By performing the action(s) on the virtual surgery, the user may rehearse the robotic surgery in the virtual surgical environment. By recording the action(s) of the user, the actual robotic surgery may be performed based on the recorded action(s) with no or minimal manual intervention.

In addition, in some embodiments, in response to an action performed by the user, more than one possible update of the configurations of the visual representations in the virtual surgery may be presented to the user to cover different possible outcomes that may occur in the actual robotic surgery. In this way, the user may foresee the different possible outcomes and prepare for the different possible outcomes in advance. Moreover, the surgery planning systems and methods disclosed herein may have high flexibility and convenience because the virtual surgery and the actual robotic surgery may be performed at any location and at any time, respectively, according to an actual need.

It should be noted that the above description regarding the process 300 is merely provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. For example, operation 360 may be omitted. As another example, the process 300 may be performed for an artificial surgery, instead of the robotic surgery.

FIG. 4 is a flowchart illustrating an exemplary process for generating a surgery video according to some embodiments of the present disclosure. In some embodiments, the process 400 may be performed by the generation module 910. In some embodiments, one or more operations of the process 400 may be performed to achieve at least part of operation 310 as described in connection with FIG. 3.

In 410, the processing device 210 may obtain multiple sets of image data relating to objects involved in the robotic surgery to be performed. For example, as shown in FIG. 4, the processing device 210 may obtain first image data 411 of a surgical robot that performs the robotic surgery, second image data 412 of a patient who receives the robotic surgery, third image data 413 of an operation room where the robotic surgery is to be performed, or the like, or any combination thereof.

In some embodiments, image data of an object involved in the robotic surgery may include 2-dimensional image data and/or 3-dimensional image data. The image data of the object may include various types of image data. For example, the second image data 412 of the patient may include medical image data and/or optical image data, etc. The medical image data may be acquired using a medical imaging device, such as an ultrasound scanning device, an X-ray scanning device, a computed tomography (CT) device, a magnetic resonance imaging (MRI) device, a positron emission scanning (PET) device, an optical coherence tomography (OCT) scanning device, and a near-infrared spectroscopy (N IRS) scanning device. The optical image data may be acquired using an optical sensor, such as, a depth camera, a structured light camera, a laser camera, a time-of-flight camera, etc.

In some embodiments, image data of different objects may be acquired by different sensors or the same sensor. Merely by way of example, the first image data 411 and/or the second image data 412 may be part of the third image data. For example, an optical sensor may capture the third image data of the operation room when the surgical robot and/or the patient are in the operation room. In such cases, the third image data 413 may include image data of the surgical robot and/or image data of the patient. The processing device 210 may generate the first image data 411 by segmenting a portion corresponding to the surgical robot from the third image data 413. Additionally or alternatively, the processing device 210 may generate the second image data 412 by segmenting a portion corresponding to the patient from the third image data 413.

In 420, for each object involved in the robotic surgery, the processing device 210 may generate a visual representation representing the object based on the image data of the object. For example, the processing device 210 may generate a virtual surgical robot 421 representing the surgical robot based on the first image data. As another example, the processing device 210 may generate a virtual patient 422 representing the patient based on the second image data. As yet another example, the processing device 210 may generate a virtual operation room 423 representing the operation room based on the third image data.

For illustration purposes, the generation of the virtual surgical robot based on the first image data is described hereinafter. In some embodiments, the processing device 210 may segment a corresponding part of the surgical robot from the first image data to generate a segmented image of the surgical robot. For example, the surgical robot may be segmented from the first image data by using a segmentation algorithm. Exemplary segmentation algorithms may include a threshold-based segmentation algorithm, a compression-based algorithm, an edge detection algorithm, a machine learning-based segmentation algorithm, or the like, or any combination thereof. In some embodiments, the surgical robot may be segmented from the first image data by using a segmentation model. The segmentation model may be trained based on a plurality of groups of training data. Each group of training data may include sample image data and a corresponding training label (e.g., a segmentation mask of the surgical robot). In some embodiments, the segmentation model may include a convolutional neural network (CNN) model, a deep CNN (DCNN) model, a fully convolutional network (FCN) model, a recurrent neural network (RNN) model, or the like, or any combination thereof.

In some embodiments, after the segmented image of the surgical robot is generated, the processing device 210 may extract a mesh surface from the segmented image of the surgical robot. The mesh surface may include a collection of vertices, edges, and faces that define a 3D shape of the surgical robot. The processing device 210 may render the mesh surface (e.g., by performing one or more visual rendering operations on the mesh surface) to generate the virtual surgical robot. In some embodiments, the processing device 210 may extract the mesh surface from the segmented image of the surgical robot by using a marching cube algorithm. In some embodiments, the mesh surface of the segmented image of each surgical robot may be a low-resolution mesh surface for faster computation in a real-time setting. The low-resolution mesh surface of the segmented image of the surgical robot may represent the virtual surgical robot by using relatively few vertexes (e.g., fewer than a threshold). In some embodiments, the visual rendering operation may include a visual transformation, a color operation, a light operation, a texture mapping operation, an animation effect operation, or the like, or any combination thereof.

In some embodiments, the processing device 210 may further combine the visual representations of the objects involved in the robotic surgery. Merely by way of example, the virtual surgical robot and the virtual patient may be placed at their respective planning positions defined in the surgery plan in the virtual operation room.

In 430, the processing device 210 may generate the surgery video displaying the process of the virtual surgery by animating at least a portion of the visual representations (e.g., the virtual surgical robot and the virtual patient) based on the surgery plan.

Taking the virtual surgical robot as an example, the processing device 210 may animate the virtual surgical robot by updating the configuration of the virtual surgical robot to perform a series of actions defined in the surgery plan. Merely by way of example, the surgery plan may include a series of actions that the surgical robot needs to perform (for example, hold a scalpel and insert it vertically into the patient at an insertion point of 2 cm below the navel, insert the scalpel into the patient for 2 centimeters, stop moving, and then drive the scalpel to move 3 centimeters in a direction toward the lower limbs of the patient, etc.). The configuration of the virtual surgical robot may be updated to perform the series of actions, and the configuration updating process may be recorded as an animation of the virtual surgical robot. In some embodiments, the configuration updating process of the virtual surgical robot may need to satisfy one or more constrains relating to the surgical robot (e.g., a degree of freedom of the robotic arm, no collision with other devices, etc.).

As another example, the processing device 210 may predict the impact of one or more surgery operations on the patient, and animate the virtual patient by updating the configuration of the virtual patient to simulate the impact. Exemplary impact of the surgery operation(s) on the patient may include patient movement, deformation of an organ, removal of an organ, bleeding, change of an emotional state, or the like, or any combination thereof. In some embodiments, the processing device 210 may predict the impact based on one or more medical images of the patient, medical psychology knowledge, medical biology knowledge, or the like, or any combination thereof.

In some embodiments of the present disclosure, a surgery video showing the process of a virtual surgery may be generated based on the surgery plan, and the user may view the surgery video and perform one or more actions on the virtual surgery to modify the surgery plan. Compared with approaches that require a user to perform the entire virtual surgery for surgery planning, the methods disclosed in the present disclosure can reduce the workload of the user and improve the efficiency of surgery planning.

It should be noted that the above description is merely provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be omitted and/or one or more additional operations may be added. For example, in operation 410, image data of one or more other objects involved in the robotic surgery, such as the surgical instrument, the sensor, etc., may be obtained. As another example, operation 430 may be omitted.

FIG. 5 is a flowchart illustrating an exemplary process for surgery planning and surgery implementation according to some embodiments of the present disclosure. As shown in FIG. 5, operations 510-530 may be performed in a surgery planning stage, and operations 540-560 may be performed in a surgery implementation stage. In some embodiments, operations 510-530 may be performed to achieve at least part of operation 330 as described in connection with FIG. 3, and operations 540-560 may be performed to achieve at least part of operation 360 as described in connection with FIG. 3.

In 510, the processing device 210 may predict possible outcomes of a target action. In some embodiments, operation 510 may be performed by the recording module 930.

As described in connection with FIG. 3, one or more actions performed by the user on the virtual surgery may be recorded. The target action may be any one of the one or more recorded actions. In some embodiments, the target action may be an action performed to achieve a specific purpose. For example, in order to achieve a purpose of cutting, the target action may be “moving a scalpel up and down.” In order to achieve the purpose of suturing, the target action may be “moving a needle forceps left and right.” In order to achieve the purpose of separation, the target action may be “holding a separation forceps and moving it to one side,” etc. In some embodiments, the target action may be a combination of a plurality of actions. For example, in order to achieve the purpose of cutting and separation, the target action may be “holding the separation forceps and moving it to one side, moving a scalpel up and down.” In some embodiments, each of the recorded action(s) may be designated as a target action, and the process 500 may be performed for each recorded action.

The possible outcomes may refer to outcomes that might occur after the target action is performed. For example, the possible outcomes may include successful removal of certain tissue, unsuccessful removal of certain tissue, organ damage, blood vessel bleeding, collision, no abnormal condition, or the like, or any combination thereof. In some embodiments, historical information relating to historical surgeries may be obtained, and reference outcomes caused by a historical action similar to the target action may be determined based on the historical information. The possible outcomes may be determined based on the reference outcomes. As another example, an outcome prediction model (e.g., a trained machine learning model) may be used to predict possible outcomes of a target action. Merely by way of example, historical actions performed in the historical surgeries and their corresponding outcomes may be collected to train the outcome prediction model. As yet another example, possible outcomes of a target action may be predicted based on human judgment. As yet another example, a possible collision between two visual representations may be predicted by analyzing the positions, the shapes, the sizes, and/or the moving trajectories of the two visual representations.

In 520, for each possible outcome, the processing device 210 may update configurations of the virtual surgical robot and the virtual patient based on the possible outcome. In some embodiments, operation 520 may be performed by the recording module 930.

For example, if the target action may cause bleeding, a certain portion of the virtual patient may be colored red to simulate the bleeding. As another example, if the target action may cause a collision between the surgical robot and the patient table, the virtual surgical robot may collide with the virtual patient table and the movement of the virtual surgical robot may be blocked by the virtual patient table. In some embodiments, taking the virtual surgical robot as an example, a lookup table including different outcomes and their corresponding configuration parameters may be previously generated. The processing device 210 may determine the configuration parameters of the virtual surgical robot corresponding to a possible outcome according to the lookup table, and update the configuration of the virtual surgical robot based on the determined configuration parameters.

In some embodiments, the updating the configuration of the virtual patient may include updating the position, the pose, the emotional state, the organ deformation, or the like, or any combination thereof, of the virtual patient. The updating of the configuration of the virtual surgical robot may include updating the position, the pose, the moving direction, the moving speed, or the like, or any combination thereof, of the virtual surgical robot.

In 530, for each possible outcome, the processing device 210 may record, based on second input received via the input component, a response action of the user to the updated configurations. In some embodiments, operation 530 may be performed by the recording module 930.

When the XR assembly displays the updated configuration(s) corresponding to a possible outcome, the user may perform one or more response actions on the virtual surgery by inputting the second user input into the input component of the XR assembly. In some embodiments, the user may input the second user input through the input component by typing, speaking, touching, drawing, etc.

A response action to a possible outcome refers to an action the user performs to deal with the possible outcome. For example, if the possible outcome includes that the target action causes bleeding, the response action of the user may include performing a hemostatic operation. As another example, if the possible outcome includes that the target action causes a collision between the surgical robot and the patient, the response action of the user may include changing the position and/or the moving direction of the virtual surgical robot, withdrawing or modifying the target action, etc. As yet another example, the user may accept the possible outcome without inputting any response action.

In 540, during the implementation of the robotic surgery, the processing device 210 may obtain monitoring information relating to the implementation of the robotic surgery. In some embodiments, operation 540 may be performed by the monitoring module 960.

The monitoring information may include any information relating to the objects involved in the robotic surgery. For example, the monitoring information may include information about the surgical robot, such as the position, the pose, and the state of the surgical robot. As another example, the monitoring information may include information about the patient, such as the position, the pose, the emotional state, the organ deformation, etc., of the patient. In some embodiments, the monitoring information may include image data, video data, audio data, operation parameters, or the like, or any combination thereof.

In some embodiments, the monitoring information may be obtained by a sensor mounted in the operation room. For example, video data may be obtained by a camera device mounted in the operation room, and audio data may be obtained by a recording device mounted in the operation room.

In 550, the processing device 210 may select, from the possible outcomes, a target outcome that actually occurs based on the monitoring information. In some embodiments, operation 550 may be performed by the monitoring module 960.

The target outcome may be a selected possible outcome that is most similar to the actual outcome occurs in the actual robotic surgery. In some embodiments, the target outcome may be selected from the possible outcomes based on the monitoring information through, for example, image recognition. For example, the monitoring information may include image data of the surgical spot of the patient captured by a camera directed to the surgical spot. Image recognition may be performed on the image data of the surgical spot to determine the actual outcome of a certain surgical action (i.e., the target outcome). In some embodiments, when an outcome that actually occurs is different from all the possible outcomes (that is, an outcome that has not been predicted occurs), an actual operation performed by the user toward the outcome may be recorded. For example, correspondence between the actual operation and the actual outcome may be recorded for providing reference information in future surgery planning (e.g., determining a possible outcome of a target action in further surgery planning).

In 560, the processing device 210 may generate a recommendation message relating to the response action performed by the user to the target outcome. In some embodiments, operation 560 may be performed by the monitoring module 960.

As aforementioned, the response actions of the user to different possible outcomes may be recorded in the surgery planning stage. In the actual surgery, if a certain possible outcome actually occurs (i.e., the certain possible outcome is the target outcome), a recommendation relating to the response action of the user to the certain outcome may be generated.

Some embodiments of the present disclosure may simulate various possible situations during surgery by predicting various possible outcomes of the target action and updating the configurations of the virtual representation(s) according to the various possible outcomes. In this way, the user may know scenarios that might occur in the actual surgery, and make preparations in advance. In addition, the response action of the user toward each possible outcome in the virtual surgery may be recorded so that based on the actual situation of the actual surgery and the previously recorded response actions, a recommendation of an appropriate action may be generated. In this way, the staffer who participates in the actual surgery can quickly take the appropriate action, and the efficiency and the accuracy of the surgery may be improved.

FIG. 6 is a flowchart illustrating an exemplary process for monitoring the implementation of a robotic surgery according to some embodiments of the present disclosure. In some embodiments, one or more operations of the process 600 may be performed to achieve at least part of operation 360 as described in connection with FIG. 3. In some embodiments, the process 600 may be performed by the monitoring module 960.

In 610, the processing device 210 may obtain monitoring information relating to the implementation of the robotic surgery. Operation 610 may be performed in a similar manner as operation 540 as described in connection with FIG. 5, and the descriptions thereof are not repeated here.

In 621, the processing device 210 may determine, based on the monitoring information, whether an action to be performed according to a modified surgery plan is risky.

In some embodiments, whether the action to be performed according to the modified surgery plan is risky may be manually determined based on the monitoring information. For example, the monitoring information and the action to be performed may be sent to a user terminal, and the staffer participating in the actual surgery may determine whether the action to be performed is risky. As another example, the processing device 210 may predict an outcome of the action to be performed based on the monitoring information, and determine whether the action is risky based on the predicted outcome. Merely by way of example, the action to be performed is moving the surgical instrument in a target direction for a target distance. The processing device 210 may determine the current positions of the surgical instrument and other objects near the surgical instrument based on the monitoring information, and predict whether the surgical instrument will collide with the other objects based on the current positions, the target direction, and the target distance. If the surgical instrument will collide with another object, the action to be performed may be deemed as being risky.

In 631, in response to determining that the action is risky, the processing device 210 may generate a notification message regarding at least one of the action or the risk associated with the action.

The notification message may include, for example, a text message, a sound message, a light message, etc. For example, a text message “The action is risky” may be displayed on a display. As another example, a warning sound may be emitted by a speaker. As yet another example, a warning light may be turned on to emit red light. In some embodiments, the notification message may be generated according to a risk level of the action to be performed. For example, the speaker may emit a louder warning sound if the action has a high risk level.

In some embodiments, the processing device 210 may cause an output device (e.g., a display, a speaker, a warning light) to output the notification message to the staffer participating in the robotic surgery. The staffer may perform corresponding action(s) in response to the notification message, for example, the staffer may cancel the action to be performed, modify the action, etc. By analyzing whether the action to be performed is risky based on the monitoring information, risk actions may be avoided and the surgery safety may be improved.

In 622, the processing device 210 may detect, based on the monitoring information, a scenario that is not included in the modified surgery plan and a corresponding action that a staffer performed in response to the scenario.

The scenario that is not included in the surgery plan may be an unanticipated scenario that occurs in the actual surgery. For example, the movement of the surgical instrument during the robotic surgery may cause an unexpected injury to an organ adjacent to the surgery site, which may be determined as a scenario that is not included in the modified surgery plan. As yet another example, the surgical robot may collide with a patient table in the robotic surgery, which may be determined as a scenario that is not included in the modified surgery plan.

When the scenario that is not included in the modified surgery plan occurs, the staffer may perform corresponding action(s) based on their own experience. For example, when the movement of the surgical instrument causes unanticipated damage (such as bleeding) to an organ near the surgical site, the staffer may perform corresponding action(s) to recover the damage. As another example, if the surgical robot collides with a patient table during the robotic surgery, the staffer may move the surgical robot to avoid the collision.

In 632, the processing device 210 may store information relating to the scenario and the corresponding action into at least one storage device.

For example, the processing device 210 may store the information relating to the scenario and the corresponding action into the storage device 220. In some embodiments, the information relating to the scenario and the corresponding action stored in the at least one storage device may be retrieved by the processing device 210 for reference in further surgery planning, for example, used for determining possible outcomes of an action.

FIG. 7 is a flowchart illustrating an exemplary process for generating a surgery plan for a surgery to be performed on a patient according to some embodiments of the present disclosure. In some embodiments, the surgery to be performed on the patient may be a robotic surgery performed by a surgical robot, an artificial surgery performed by a staffer, or a mixed surgery performed by the robotic surgery and the staffer together. In some embodiments, one or more operations of the process 700 may be performed to achieve at least part of operation 310 as described in connection with FIG. 3. In some embodiments, the process 700 may be performed by the generation module 910.

In 710, the processing device 210 may generate a virtual operation room representing an operation room where the surgery is to be performed. The virtual operation room may include one or more visual representations representing one or more objects located in the operation room during the surgery. For example, the visual representations may include a virtual surgical robot representing a surgical robot, a virtual patient representing a patient, a virtual sensor representing a sensor, a virtual staffer representing a staffer, a virtual surgical instrument representing a surgical instrument.

More descriptions regarding the generation of the virtual operation room and the visual representations in the virtual operation room may be found elsewhere in the present disclosure. See, e.g., FIG. 4 and relevant descriptions thereof.

In 720, the processing device 210 may determine one or more optimization targets relating to the one or more visual representations.

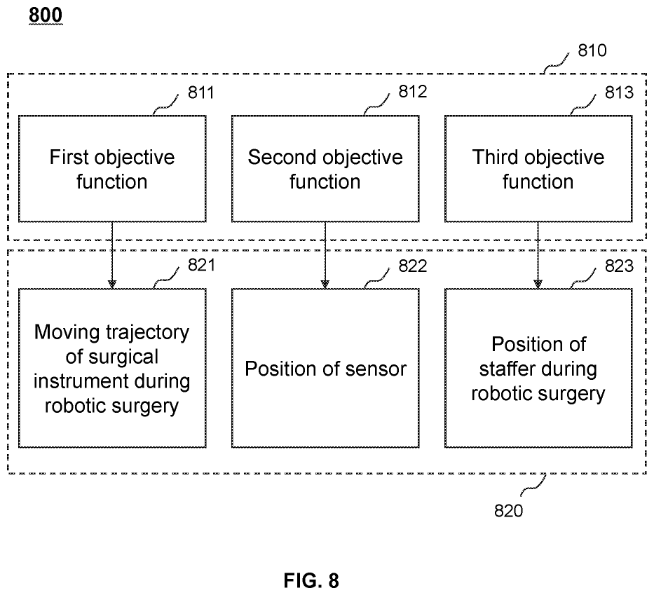

The optimization target(s) may relate to various aspects of the surgery, such as a position of the staffer, a position of the surgical instrument, a position of the surgical robot, a surgery procedure, a moving trajectory of the surgical instrument (e.g., an insertion point, an insertion angle, etc., of the surgical instrument), a movement of the surgical robot, etc. In some embodiments, the optimization target(s) may be expressed as one or more objective functions. The objective function(s) may include a loss function, an optimization function, a constraint, etc. More descriptions regarding the objective function may be found elsewhere in the present disclosure. See, e.g., FIG. 8 and relevant descriptions thereof.

In some embodiments, the one or more optimization targets may include a plurality of optimization targets that have their respective priorities. The priority of an optimization target may indicate an importance of the optimization target. More attention may need to be paid to satisfy an optimization target having a high priority than an optimization target having a low priority. Merely by way of example, an optimization target relating to the moving trajectory of the surgical instrument may have a higher priority than an optimization target relating to the position of the surgical robot.

In some embodiments, the processing device 210 may determine the optimization target based on an input of the user. For example, the user may determine the patient's condition based on the patient's information, and then determine an optimization target relating to the patient. The user may input the optimization target into the surgery planning system by an input component (e.g., the input component 132). In some embodiments, the processing device 210 may determine the optimization target according to preset rules. For example, the preset rules may specify different optimization targets corresponding to different patients, different optimization targets corresponding to different surgical protocols, etc. The processing device 210 may determine the optimization target according to the patient type, the surgical protocol of the surgery to be performed, and the preset rules. In some embodiments, the preset rules may be determined in advance. The processing device 210 may update the preset rules according to an instruction of the user.

In 730, the processing device 210 may generate the surgery plan by optimizing configurations of the one or more visual representations to meet at least part of the one or more optimization targets.

In some embodiments, the optimization of the configurations of the visual representations may be performed by updating the configurations of the visual representations until the at least part of the optimization target(s) is met. In some embodiments, the updating of the configuration of a visual representation may include updating the location, the pose, the state, the moving direction, the orientation, or the like, or any combination thereof, of the visual representation.

In some embodiments, the processing device 210 may update the configurations of the visual representation(s) multiple times, and then determine whether the optimization target is met after each time the configurations are updated. In some embodiments, after each updating of the configuration(s), the user can provide additional information or an instruction to guide the next step of the configuration updating. In some embodiments, after each updating of the configuration(s), the processing device 210 may proactively ask the user for additional information to determine the strategies for the next step of the configuration updating.

In some embodiments, multiple optimization targets may be jointly optimized. Alternatively, multiple optimization targets may be sequentially optimized based on a predefined order, or a randomly determined order, or an automatically determined order.

For illustration purposes, FIG. 8 provides a schematic diagram illustrating exemplary optimization targets according to some embodiments of the present disclosure.

As shown in FIG. 8, the optimization targets may be represented by one or more objective functions 810. The objective function 810 may include a first objective function 811, a second objective function 812, and a third objective function 813.

The first objective function 811 may relate to a deviation of a moving trajectory of the virtual surgical instrument from a planning surgical route. For example, the optimization targets may include a first optimization target that the first objective function 811 is lower than a first threshold value or has a local minimum value.

In some embodiments, the processing device 210 may determine the planning surgical route based on a medical image (e.g., a CT image, an MRI image) of the patient. For example, the processing device 210 may identify the surgical site from the medical image through image recognition (e.g., an image recognition model), and determine a preliminary route in the image domain from the body surface of the patient to the surgical site. In the determination of the preliminary route, one or more constraints, e.g. critical organ(s) near the surgical site need to be avoided, the length of the preliminary route needs to be short if possible, etc., may be taken into consideration. The processing device 210 may further transform the preliminary route in the image domain to the surgical route based on a transformation relationship between an imaging coordinate system and a surgical coordinate system.

In some embodiments, the processing device 210 may optimize configurations of the one or more visual representations to meet the first optimization target. For example, the processing device 210 may obtain one or more constraint(s) associated with the surgical robot and the surgical instrument. Merely by way of example, the constraint(s) may include the count of robotic arms of the surgical robot, the degrees of freedom for each robotic arm, a potential collision between the robotic arms, a potential collision between a robotic arm and the patient, other potential collisions (e.g. with an anesthesia equipment or a patient table), or the like, or any combination thereof. In some embodiments, the constraint(s) may be manually set by the user, or determined based on the system default settings, or determined by the processing device 210 (for example, based on the surgical robot configuration).

In some embodiments, the processing device 210 may generate a plurality of possible moving trajectories of the virtual surgical instrument by updating the configurations of the virtual surgical robot and the virtual surgical instrument based on the one or more constraints. For example, the processing device 210 may deform the virtual robotic arms of the virtual surgical robot under the constrain of the degree of freedom of the virtual robotic arms to move the virtual surgical instrument without causing any collision, wherein the moving trajectory of the virtual surgical instrument may be designate as a possible moving trajectory.

Further, the processing device may determine a value of the first objective function 811 corresponding to each of the plurality of possible moving trajectories. For example, for each possible moving trajectory, the processing device 210 may determine the deviation of the possible moving trajectory from the planning surgical route, and determine the value of the first objective function 811 based on the deviation. The smaller the deviation, the smaller the value of the first objective function 811 may be.

The processing device 210 may then determine a moving trajectory 821 of the surgical instrument in the robotic surgery based on the value of the first objective function 811 of the possible moving trajectories. In some embodiments, the processing device 210 may select, from the plurality of possible moving trajectories, one or more possible moving trajectories whose corresponding values of the first objective function 811 satisfy a first preset condition. The processing device 210 may further determine the moving trajectory of the surgical instrument during the robotic surgery based on one or more selected possible moving trajectories. Merely by way of example, the first preset condition may be that the corresponding value of the first objective function 811 is the smallest, the corresponding value of the first objective function 811 is lower than the first threshold value, etc. In some embodiments, if there are multiple selected possible moving trajectories, the processing device 210 or the user may designate one from the selected possible moving trajectories as the moving trajectory 821 of the surgical instrument. Alternatively, the processing device 210 may determine the moving trajectory from the selected possible moving trajectories based on, for example, the length, the effect on adjacent organs, etc., of each selected possible moving trajectory.

In some embodiments, the processing device 210 may further determine the position of the surgical robot according to the moving trajectory of the surgical instrument. For example, the processing device 210 may determine the position of the surgical robot according to the insertion point at which the surgical instrument is inserted into the human body and a constraint of a shape of the surgical robot (e.g., a degree of freedom of the robotic arm, etc.).

In some embodiments, the one or more visual representations may include a virtual sensor representing a sensor mounted in the operation room.

The second objective function 812 may relate to a coverage rate of a target area in the virtual operation room by a field of view (FOV) of the virtual sensor. For example, the optimization targets may include a second optimization target that the second objective function 812 is higher than a second threshold value or has a local maximum value. The target area may correspond to an area that needs to be monitored in the operation room, for example, an area where the patient is located (especially the portion of the patient that receives the surgery), an area where the surgical robot is located, an area where the surgical instrument is located, etc. In some embodiments, different target areas may correspond to different virtual sensors. The different virtual sensors may be located at different positions in the virtual operation room.

In some embodiments, the processing device 210 may optimize the configuration of the virtual sensor to meet the second optimization target. For example, the processing device 210 may update the configuration of the virtual sensor to determine a plurality of possible sensor positions of the virtual sensor in the virtual operation room. The virtual sensor may be located in any position in the virtual surgery, such as a wall, a ceiling, etc., of the virtual operation room. The virtual sensor may also be mounted on other visual representations in the virtual operation room. For example, the virtual sensor may be mounted on the virtual surgical robot, etc. In some embodiments, the processing device 210 may determine the plurality of possible sensor positions in various ways. For example, the processing device may randomly determine the plurality of possible sensor positions. As another example, the user may input the plurality of possible sensor positions via an input component.

In some embodiments, the processing device may determine a value of the second objective function 812 corresponding to each of the plurality of possible sensor positions. The value of the second objective function 812 corresponding to a possible sensor position may be proportional to the coverage rate of the virtual sensor at the possible sensor position. For example, the greater the coverage rate, the higher the value of the second objective function 812. The processing device 210 may further determine a position 822 of the sensor based on the values of the second objective function 812.

In some embodiments, the processing device 210 may select, from the plurality of possible sensor positions, one or more possible sensor positions whose corresponding values of the second objective function 812 satisfy a second preset condition. The processing device 210 may further determine the position of the sensor during the robotic surgery based on one or more selected possible sensor positions. Merely by way of example, the second preset condition may be that the corresponding value of the second objective function 812 is the largest, the corresponding value of the second objective function 812 is greater than the second threshold value. For example, the processing device may designate a possible sensor position corresponding to the largest value of the second objective function 812 among the plurality of possible sensor positions as the position 822 of the sensor. The largest value of the second objective function 812 among the plurality of possible sensor positions may be regarded as the local maximum value of the second objective function 812.

In some embodiments, if there are multiple selected possible sensor positions, the processing device 210 or the user may designate one from the selected possible sensor position as the position 822 of the sensor. Alternatively, the processing device 210 may determine the position 822 of the sensor from the selected possible sensor positions based on, for example, the user preference, the imaging quality of the sensor at each possible sensor position, etc.

In some embodiments, the one or more visual representations may include a virtual staffer representing a staffer who assists the robotic surgery and a virtual medical device representing a medical device (e.g., a medical imaging device) configured to emit radiation toward the patient during the robotic surgery. The third objective function 813 may relate to a radiation dosage received by the staffer during the robotic surgery. For example, the optimization targets may include a third optimization target that the third objective function 813 is lower than a third threshold value or has a local minimum value.

In some embodiments, the processing device 210 may update the configuration of the virtual staffer to determine a plurality of possible staffer positions relative to the virtual medical device. The determination of the possible staffer positions may be performed in a similar manner as that of the possible sensor positions, and the descriptions thereof are not repeated here.

For each possible staffer position, the processing device 210 may determine a value of the third objective function 813. In some embodiments, the processing device 210 may determine a distance between a possible staffer position and a radiation source of the medical device, and determine the value of the third objective function 813 corresponding to the possible staffer position based on the distance. If the distance is short, the staffer may receive a high radiation dosage at the possible staffer position, and the corresponding value of third objective function 813 may be large.

In some embodiments, the processing device 210 may determine the position 823 of the staffer based on the third objective function 813. In some embodiments, the processing device 210 may select, from the plurality of possible staffer positions, one or more possible staffer positions whose corresponding values of the third objective function 813 satisfy a third preset condition. The processing device 210 may further determine the position 823 of the staffer during the robotic surgery based on one or more selected possible staffer positions. Merely by way of example, the third preset condition may be that the corresponding value of the third objective function 813 is the smallest, the corresponding value of the third objective function 813 is lower than the third threshold value. In some embodiments, if there are multiple selected possible staffer positions, the processing device 210 or the user may designate one from the selected possible staffer positions as the position 823 of the staffer. Alternatively, the processing device 210 may determine the position 823 from the selected possible staffer positions based on, for example, the user preference, the distance between each selected possible staffer position to the patient, etc.

In some embodiments of the present disclosure, the processing device 210 may designate the possible staffer position corresponding to the smallest value of the third objective function 813 as the position of the staffer, so that the radiation dosage received by the staffer during the robotic surgery may be reduced.

In some embodiments of the present disclosure, the surgery plan may be generated by optimizing configurations of the one or more visual representations to meet the one or more optimization targets, which can ensure the accuracy of the surgery plan. Especially, the configuration updating of the visual representations may be performed based on the XR technique in the virtual surgical environment, which is more intuitive, realistic, and accurate compared with conventional surgery planning techniques performed by pure data manipulation and calculation. Moreover, through the XR technique, the user may have a more intuitive perception of the surgery planning process and provide feedback information for the surgery planning.

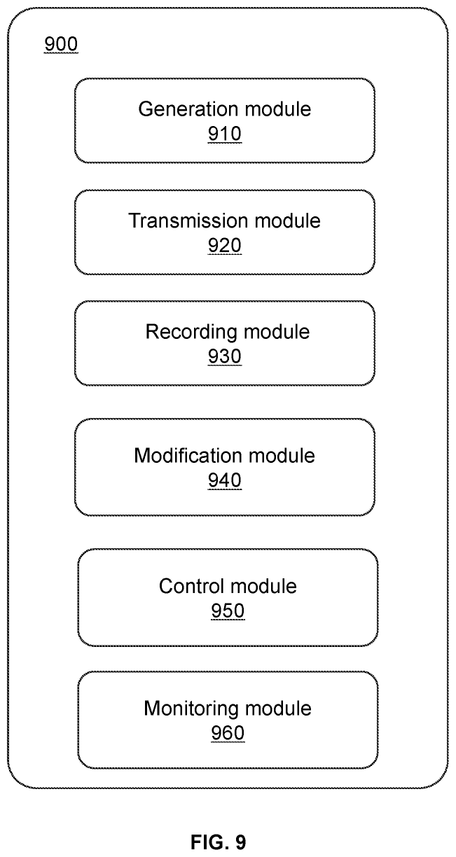

FIG. 9 is a block diagram illustrating an exemplary processing device according to some embodiments of the present disclosure. As shown in FIG. 9, the processing device 210 may include a generation module 910, a transmission module 920, a recording module 930, a modification module 940, a control module 950, and a monitoring module 960.

The generation module 910 may be configured to generate, based on a surgery plan relating to a robotic surgery to be performed on a patient by a surgical robot, a surgery video displaying a process of a virtual surgery performed by a virtual surgical robot on a virtual patient. More descriptions regarding the generation of the surgery video may be found elsewhere in the present disclosure. See, e.g., operation 310 and relevant descriptions thereof.

The transmission module 920 may be configured to transmit the surgery video to a display component of an XR assembly used to render the surgery video for display to a user. More descriptions regarding the transmission of the surgery video may be found elsewhere in the present disclosure. See, e.g., operation 320 and relevant descriptions thereof.

The recording module 930 may be configured to record, based on user input received via an input component of the XR assembly, one or more actions that the user performed on the virtual surgery. More descriptions regarding the recording of the action(s) may be found elsewhere in the present disclosure. See, e.g., operation 330 and relevant descriptions thereof.

The modification module 940 may be configured to modify the surgery plan based on the one or more recorded actions. More descriptions regarding the modification of the surgery plan may be found elsewhere in the present disclosure. See, e.g., operation 340 and relevant descriptions thereof.

The control module 950 may be configured to cause the surgical robot to implement the robotic surgery on the patient according to the modified surgery plan. More descriptions regarding the implementation of the robotic surgery may be found elsewhere in the present disclosure. See, e.g., operation 350 and relevant descriptions thereof.

The monitoring module 960 may be configured to monitor the implementation of the robotic surgery. More descriptions regarding the monitoring of the robotic surgery may be found elsewhere in the present disclosure. See, e.g., operation 360 and relevant descriptions thereof.

In some embodiments, the processing device 210 may include one or more additional modules and/or one or more modules of the processing device 210 described above may be omitted. Additionally or alternatively, two or more modules of the processing device 210 may be integrated into a single module. A module of the processing device 210 may be implemented on two or more sub-modules.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer-readable media having computer-readable program code embodied thereon.

A non-transitory computer-readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer-readable signal medium may be any computer-readable medium that is not a computer-readable storage medium and that may communicate, propagate, or transport a program for use by or in connection with an instruction execution system, apparatus, or device. Program code embodied on a computer-readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object-oriented programming language such as Java, Scala, Smalltalk, Eiffel, JADE, Emerald, C++, C #, VB. NET, Python, or the like, conventional procedural programming languages, such as the “C” programming language, Visual Basic, Fortran, Perl, COBOL, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A system comprising:
at least one storage device storing a set of instructions for surgery planning; and
at least one processor configured to communicate with the at least one storage device, wherein when executing the set of instructions, the at least one processor is configured to direct the system to perform operations including:
generating, based on a surgery plan relating to a robotic surgery to be performed on a patient by a surgical robot, a surgery video displaying a process of a virtual surgery performed by a virtual surgical robot on a virtual patient;
transmitting the surgery video to a display component of an XR assembly used to render the surgery video for display to a user;
recording, based on user input received via an input component of the XR assembly, one or more actions that the user performed on the virtual surgery;
modifying the surgery plan based on the one or more recorded actions; and
causing the surgical robot to implement the robotic surgery on the patient according to the modified surgery plan.

2. The system of claim 1, wherein the generating a surgery video displaying a process of a virtual surgery comprises:
obtaining first image data of the surgical robot and second image data of the patient;
generating the virtual surgical robot representing the surgical robot based on the first image data;
generating the virtual patient representing the patient based on the second image data; and
generating the surgery video displaying the process of the virtual surgery by animating the virtual surgical robot and the virtual patient based on the surgery plan.

3. The system of claim 2, wherein the surgery video further displays an operation room where the robotic surgery is to be performed, and the generating a surgery video displaying a process of a virtual surgery comprises:
obtaining third image data of the operation room captured by one or more sensors; and
generating a virtual operation room representing the operation room based on the third image data, wherein in the virtual video, the virtual surgical robot and the virtual patient are placed at their respective planning positions specified in the surgery plan in the virtual operation room.

4. The system of claim 1, wherein the recording, based on user input received from an input component of the XR assembly, one or more actions that the user performed on the virtual surgery comprises:
determining, based on the user input, one or more candidate actions that the user intends to perform on the virtual surgery;
for each of the one or more candidate actions,
updating configurations of the virtual surgical robot and the virtual patient in the surgery video in response to the candidate action; and
recording the candidate action as one of the one or more actions in response to determining that the updated configurations satisfy a preset condition.

5. The system of claim 1, wherein the operations further include:
for a target action of the one or more actions predicting possible outcomes of the target action;
for each of the possible outcomes,
updating configurations of the virtual surgical robot and the virtual patient based on the possible outcome; and
recording, based on second user input received via the input component, a response action of the user to the updated configurations.

6. The system of claim 5, wherein the operations further include:
during the implementation of the robotic surgery:
obtaining monitoring information relating to the implementation of the robotic surgery;
selecting, from the possible outcomes, a target outcome that actually occurs based on the monitoring information; and
generating a recommendation relating to the response action of the user to the target outcome.

7. The system of claim 1, wherein the operations further include:
during the implementation of the robotic surgery:
obtaining monitoring information relating to the implementation of the robotic surgery;
determining, based on the monitoring information, whether an action to be performed according to the modified surgery plan is risky;
in response to determining that the action is risky, generating a notification regarding at least one of the action or a risk associated with the action.

8. The system of claim 1, wherein the XR assembly is operably connected to the at least one processor via a first network, the surgical robot is operably connected to the at least one processor via a second network, and at least one of the first network or the second network includes a wireless network.

9. The system of claim 1, wherein the operations further comprise generating the surgery plan by:
generating a virtual operation room representing an operation room where a surgery is to be performed, the virtual operation room including one or more visual representations, the visual representations including the virtual surgical robot and the virtual patient;
determining one or more optimization targets relating to the one or more visual representations; and
generating the surgery plan by optimizing configurations of the one or more visual representations to meet at least part of the one or more optimization targets.

10. The system of claim 9, wherein the one or more visual representations further include a virtual surgical instrument that represents a surgical instrument and is operably coupled to the virtual surgical robot,
the one or more optimization targets include an objective function that relates to a deviation of a moving trajectory of the virtual surgical instrument from a planning surgical route, and optimizing configurations of the one or more visual representations to meet at least part of the one or more optimization targets comprises:
obtaining one or more constraints relating to the surgical robot and the surgical instrument;
updating configurations of the virtual surgical robot and the virtual surgical instrument based on the one or more constraints to generate a plurality of possible moving trajectories of the virtual surgical instrument;
determining a value of the objective function corresponding to each of the plurality of possible moving trajectories;
selecting, from the plurality of possible moving trajectories, one or more possible moving trajectories whose corresponding values of the objective function satisfy a preset condition; and
determining, based on one or more selected possible moving trajectories, a moving trajectory of the surgical instrument during the robotic surgery.

11. The system of claim 10, wherein the operations further comprise:
determining a position of the surgical robot based on the moving trajectory of the surgical instrument.

12. The system of claim 9, wherein the one or more visual representations further include a virtual sensor representing a sensor, and the one or more optimization targets include an objective function that relates to a coverage rate of a target area in the virtual operation room by a field of view (FOV) of the virtual sensor, and optimizing configurations of the one or more visual representations to meet at least part of the one or more optimization targets comprises:
updating a configuration of the virtual sensor to determine a plurality of possible sensor positions of the sensor;
determining a value of the objective function corresponding to each of the plurality of possible sensor positions;
selecting, from the plurality of possible sensor positions, one or more possible sensor positions whose corresponding values of the objective function satisfy a preset condition; and
determining, based on the one or more selected possible sensor positions, a position of the sensor.

13. The system of claim 9, wherein the one or more visual representations further includes a virtual staffer representing a staffer who assists the robotic surgery and a virtual medical device representing a medical device configured to emit radiation toward the patient during the robotic surgery,
the one or more optimization targets include an objective function that relates to a radiation dosage received by the staffer during the robotic surgery, and
optimizing configurations of the one or more visual representations to meet at least part of the one or more optimization targets are met comprises:
updating a configuration of the virtual staffer to determine a plurality of possible staffer positions relative to the virtual medical device;
determining a value of the objective function corresponding to each of the plurality of possible staffer positions;
selecting, from the plurality of possible staffer positions, one or more possible staffer positions whose corresponding values of the objective function satisfy a preset condition; and
determining, based on the one or more selected possible staffer positions, a position of the staffer during the robotic surgery.

14. A system comprising:
at least one storage device storing a set of instructions for surgery planning; and
at least one processor configured to communicate with the at least one storage device, wherein when executing the set of instructions, the at least one processor is configured to direct the system to perform operations including:
generating a virtual operation room representing an operation room where a surgery is to be performed, the virtual operation room including one or more visual representations representing one or more objects located in the operation room during the surgery;
determining one or more optimization targets relating to the one or more visual representations; and
generating a surgery plan with respect to the surgery by optimizing configurations of the one or more visual representations to meet at least part of the one or more optimization targets.

15. A method for surgery planning implemented on a computing device having at least one processor and at least one storage device, the method comprising:
generating, based on a surgery plan relating to a robotic surgery to be performed on a patient by a surgical robot, a surgery video displaying a process of a virtual surgery performed by a virtual surgical robot on a virtual patient;
transmitting the surgery video to a display component of an XR assembly used to render the surgery video for display to a user;
recording, based on user input received via an input component of the XR assembly, one or more actions that the user performed on the virtual surgery;
modifying the surgery plan based on the one or more recorded actions; and
causing the surgical robot to implement the robotic surgery on the patient according to the modified surgery plan.

16. The method of claim 15, wherein the generating a surgery video displaying a process of a virtual surgery comprises:
obtaining first image data of the surgical robot and second image data of the patient;
generating the virtual surgical robot representing the surgical robot based on the first image data;
generating the virtual patient representing the patient based on the second image data; and
generating the surgery video displaying the process of the virtual surgery by animating the virtual surgical robot and the virtual patient based on the surgery plan.

17. The method of claim 16, wherein the surgery video further displays an operation room where the robotic surgery is to be performed, and the generating a surgery video displaying a process of a virtual surgery comprises:
obtaining third image data of the operation room captured by one or more sensors; and
generating a virtual operation room representing the operation room based on the third image data, wherein in the virtual video, the virtual surgical robot and the virtual patient are placed at their respective planning positions specified in the surgery plan in the virtual operation room.

18. The method of claim 15, wherein the recording, based on user input received from an input component of the XR assembly, one or more actions that the user performed on the virtual surgery comprises:
determining, based on the user input, one or more candidate actions that the user intends to perform on the virtual surgery;
for each of the one or more candidate actions,
updating configurations of the virtual surgical robot and the virtual patient in the surgery video in response to the candidate action; and
recording the candidate action as one of the one or more actions in response to determining that the updated configurations satisfy a preset condition.

19. The method of claim 15, wherein the method further comprises:
for a target action of the one or more actions predicting possible outcomes of the target action;
for each of the possible outcomes,
updating configurations of the virtual surgical robot and the virtual patient based on the possible outcome; and
recording, based on second user input received via the input component, a response action of the user to the updated configurations.

20. The method of claim 15, wherein the method further comprises:
during the implementation of the robotic surgery:
obtaining monitoring information relating to the implementation of the robotic surgery;
determining, based on the monitoring information, whether an action to be performed according to the modified surgery plan is risky;
in response to determining that the action is risky, generating a notification regarding at least one of the action or a risk associated with the action.
